(19) United States
(12) Patent Application Publication
(10) Pub. No.: US 2018/0132716 A1
(43) Pub. Date: May 17, 2018

(54) DEVICE FOR MEASURING INTRA-OCULAR PRESSURE

(71) Applicant: David P. AVIRAM, Kingston Upon Thames (London) (GB)

(72) Inventor: David P. AVIRAM, Kingston Upon Thames (London) (GB)

(21) Appl. No.: 15/726,482

(22) PCT Filed: Apr. 8, 2016
(86) PCT No.: PCT/GB2016/000077

§ 371 Date: Oct. 6, 2017

(30) Foreign Application Priority Data
Apr. 8, 2015 (GB) 1505995.9

(51) Int. Cl. A61B 3/16; A61B 3/00; A61M 1/00

(52) U.S. Cl. CPC A61B 3/16; A61B 3/0008; A61M 2210/0612; A61B 2562/0261; A61M 1/0088

(57) ABSTRACT

A device for non-invasive determination of intra-ocular pressure comprising a pressure chamber with a flexible membrane for engaging with the cornea of a subject's eye and a strain gauge, wherein the pressure chamber may be internally pressurised and thereby exerts a pressure via the membrane upon both the cornea and the strain gauge to that result in a deflection of the cornea and strains being generated at the strain gauge, which, in turn, enable to establish a correlation between inter ocular pressure and strains.

The invention relates to a device for measuring Intra ocular pressure.

Measurement and detection of elevated Intra ocular pressures is a prime method for early detection of the onset of glaucoma.

Current methods for non-invasive detection of intra ocular pressures io include the Goldman Aplanation method that involves the measurement of reactive forces due to a plunger, of known cross sectional area, engaging and flattening a small area of the cornea. Other methods involve the use of a jet of air for measuring the resulting corneal deformation. The main deficiencies of these methods arise from inaccuracies due to poor alignment with the radial axis of the cornea; the variation in local stiffness between cornea and the sclera and errors due to local deformation of a small area of the cornea when subjected to an external force.

Such constraints are known to have generated unreliable data about the actual pressure within the ocular region of the eye.

It is an aim of the present invention to provide an improved device for measuring inter ocular pressure. Another aim is to provide a solution of a type that is not made in a conventional manner and that will overcome the limitations mentioned above.

The invention provides a device for measuring inter ocular pressure as defined by claim 1 of the set of claims following this description. Preferred and/or optional features of the invention are the subject of other claims of the set of claims.

An alternative solution for measuring intra ocular pressure that overcomes the above mentioned constraints is the main aim of this invention and will hereby be described by means of a non-limited example.

Accordingly, this invention provides for a pressure measuring device, characterized in that it has a pressure chamber that is equipped with a flexible membrane for contacting the cornea via an engagement contact lens and a strain measuring piece, on the opposite side of the pressure chamber, for measuring strains. Deflections of the cornea are dependent upon the difference between the applied external chamber pressure and the intraocular pressure. By using metered volumes of fluid to pressurise the chamber, a correlation between applied pressure and strains can be established. Hence, a comparison between strains and a range of applied to pressures may be used to enable the determination of a specific range of inter ocular pressures. Results so obtained may be used to provide several data points that reduce errors associated with using a single data point as is the current practice. By such means it is possible to overcome errors due to directionality and local deformations, as mentioned above.

In one preferred embodiment of a pressure measuring device, a contact lens engagement piece may be used for providing hygienic separation between the cornea and the flexible membrane of the pressure chamber. The strain measuring piece may be placed or form part of the flexible membrane and thus be directly affected by pressurisation from within the pressure chamber. In turn, deflections of the cornea will result in corresponding strains at the strain measuring piece. Thus, a correlation between strains and inter ocular pressure may be established.

One embodiment of the invention will be described solely by way of non-limited example and with reference to the accompanying drawings in which:

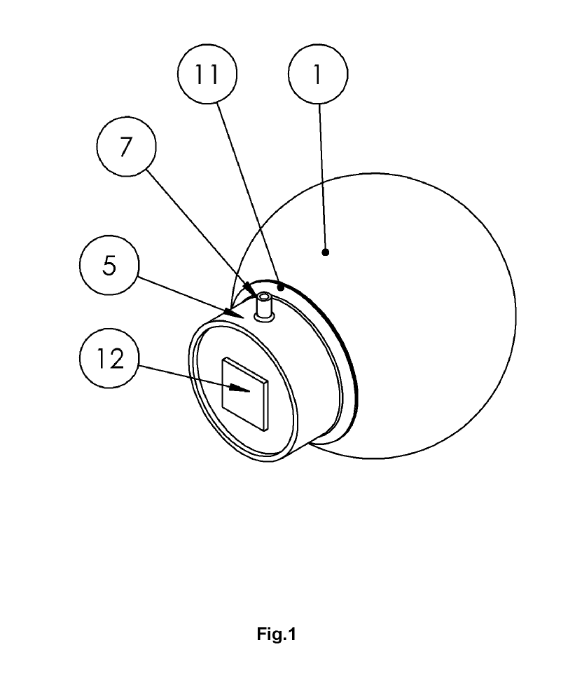

FIG. 1 shows a general external view of a pressure measuring device in contact with an eye;

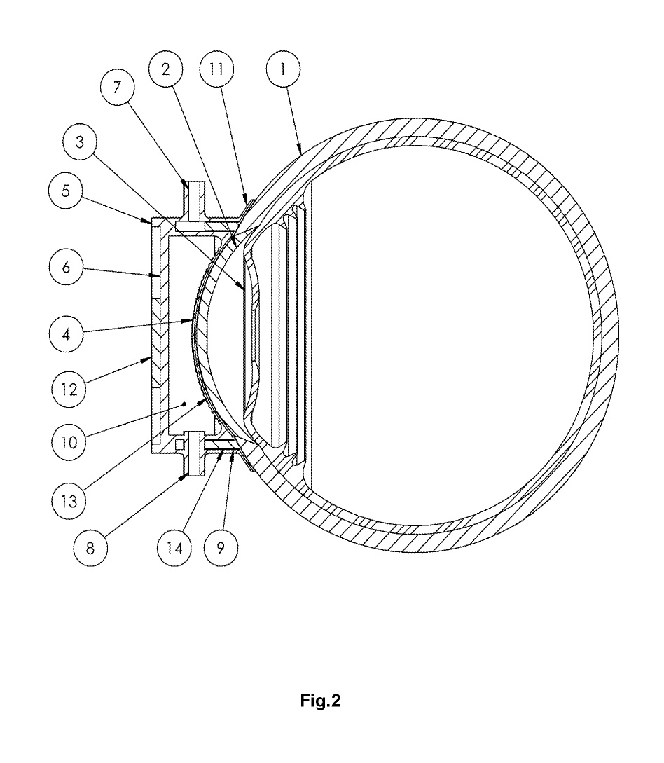

FIG. 2 shows a sectional view of a pressure measuring device attached to the eye;

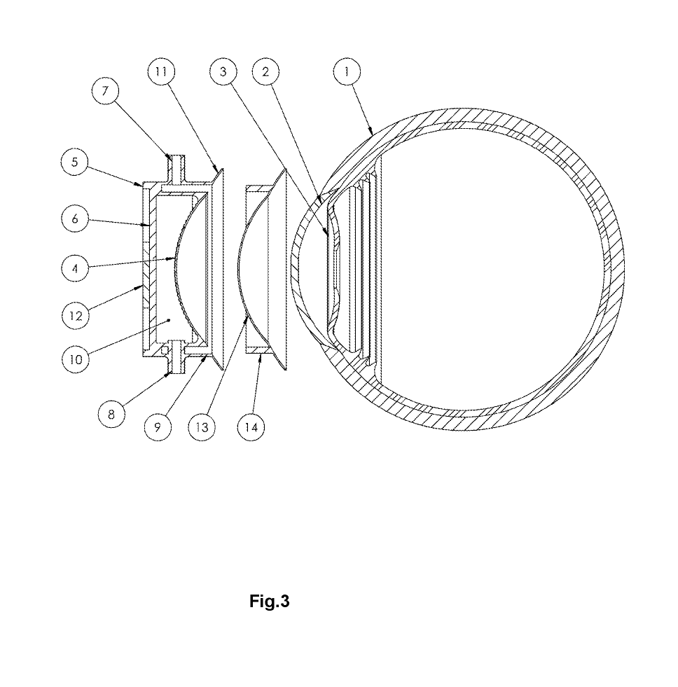

FIG. 3 shows a sectional exploded view of a pressure measuring device near the eye;

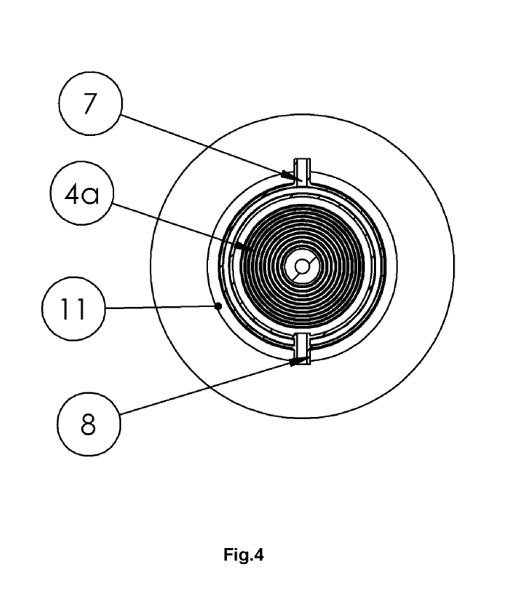

FIG. 4 shows a vertical section across the pressure chamber;

FIG. 1 shows an assembly of a pressure measuring device. The assembly consists of pressure chamber (5), with external suction port (7) and strain measurement piece (12), attached to sclera (1) via engagement ring (11).

FIG. 2 shows a vertical sectional view of an assembly of a pressure measuring device consisting of sclera (1), cornea (2) and regions of an eye. The pressure measuring device (5) has pressure chamber (10) that is equipped with strain measuring piece (12) and transparent piece (6). The pressure within the chamber (10) may be varied by means of pressure port (8). The flexible membrane (4) is held onto the sclera (1) by means of engagement ring (11) that has a peripheral suction channel (9), which is connected to an external suction port (7). The cornea (2) is in contact with contact lens engagement piece (13) that has ring (14) for axial alignment and engagement with peripheral suction channel (9). The contact lens engagement piece (13) is in contact with flexible membrane (4) and the cornea (2) which, in turn, is subjected to pressurisation from chamber (10). Transparent piece (6) enables investigation of the contours of the iris (3) by means of ultrasound and interferometry.

FIG. 3 shows a sectional exploded view of a pressure measuring device.

FIG. 4 shows a vertical section across the pressure chamber showing pressure port (8), suction port (7), strain gauge (4a) and engagement ring (11).

1. A device assembly for non-invasive determination of intra-ocular pressure comprising at least one pressure chamber with a flexible membrane, wherein the pressure chamber has an inlet port for charging the chamber with fluid and exerting pressure on a cornea via the flexible membrane and on a strain measurement means for determination of inter-ocular pressure due to deflection of the cornea.

2. A The device assembly of claim 1, wherein the flexible membrane has a strain gauge attached to the outer surface of the flexible membrane for determination of strains due to internal pressure variations within the pressure chamber.

3. The device assembly of claim 1, which further comprises a measurement piece having patterns that enable interrogation by means of interferometry for determination of strains due to internal pressure variations within the pressure chamber.

4. The device assembly of claim 3, wherein the flexible membrane is transparent for enabling electro-magnetic radiation, which may optionally comprise visible light, to pass through the membrane and the measurement piece for interrogation by external means.

5. The device assembly of claim 1, wherein contact between flexible membrane and the cornea is enhanced by means of contact between a sclera and an engagement ring which is equipped with a peripheral suction port and which, in turn, is guided by means of a ring.

6. The device assembly of claim 1, which further comprises a lens engagement piece which, in use, is located between the flexible membrane and the cornea.
